SEQUENCE LISTING (128 sequences)

SEQ ID NO: 1 - DNA, 24 bp, Artificial Sequence
  Synthesized
tgctgcttttgtgcttttgtgctt

SEQ ID NO: 2 - DNA, 24 bp, Artificial Sequence
  Synthesized
tcatcattttgtcattttgtcatt

SEQ ID NO: 3 - DNA, 24 bp, Artificial Sequence
  Synthesized
tcctccttttgtccttttgtcctt

SEQ ID NO: 4 - DNA, 24 bp, Artificial Sequence
  Synthesized
tcttctttttgtctttttgtcttt

SEQ ID NO: 5 - DNA, 24 bp, Artificial Sequence
  Synthesized
tagtagttttgtagttttgtagtt

SEQ ID NO: 6 - DNA, 24 bp, Artificial Sequence
  Synthesized
tggtggttttgtggttttgtggtt

SEQ ID NO: 7 - DNA, 24 bp, Artificial Sequence
  Synthesized
ttgttgttttgttgttttgttgtt

SEQ ID NO: 8 - DNA, 24 bp, Artificial Sequence
  Synthezied
tgctgcaaaagagcaaaagagcaa

SEQ ID NO: 9 - DNA, 24 bp, Artificial Sequence
  Synthesized
tgctgcccccgcgcccccgcgccc

SEQ ID NO: 10 - DNA, 24 bp, Artificial Sequence
  Synthesized
tcatttttttgtttttttgtcatt

SEQ ID NO: 11 - DNA, 24 bp, Artificial Sequence
  synthesized
tcattgttttgttgttttgtcatt

SEQ ID NO: 12 - DNA, 24 bp, Artificial Sequence
  synthesized
tcattcttttgttcttttgtcatt

SEQ ID NO: 13 - DNA, 24 bp, Artificial Sequence
  synthesized
aaaaaactaaaaaaaactaaaaaa

SEQ ID NO: 14 - DNA, 24 bp, Artificial Sequence
  synthesized
tcataattttgtaattttgtcatt

SEQ ID NO: 15 - DNA, 24 bp, Artificial Sequence
  synthesized
tcattattttgttattttgtcatt

SEQ ID NO: 16 - DNA, 24 bp, Artificial Sequence
  synthesized
tcatgattttgtgattttgtcatt

SEQ ID NO: 17 - DNA, 24 bp, Artificial Sequence
  synthesized
tcatccttttgtccttttgtcatt

SEQ ID NO: 18 - DNA, 24 bp, Artificial Sequence
  synthesized
tcatctttttgtctttttgtcatt

SEQ ID NO: 19 - DNA, 24 bp, Artificial Sequence
  SYNTHESIZED
tttttttttttttttttttttttt

SEQ ID NO: 20 - DNA, 24 bp, Artificial Sequence
  SYNTHESIZED
cattttgttttttttttttttttt

SEQ ID NO: 21 - DNA, 24 bp, Artificial Sequence
  SYNTHESIZED
ttcattttgttttttttttttttt

SEQ ID NO: 22 - DNA, 24 bp, Artificial Sequence
  SYNTHESIZED
ttttcattttgttttttttttttt

SEQ ID NO: 23 - DNA, 24 bp, Artificial Sequence
  SYNTHESIZED
ttttttcattttgttttttttttt

SEQ ID NO: 24 - DNA, 24 bp, Artificial Sequence
  SYNTHESIZED
ttttttttcattttgttttttttt

SEQ ID NO: 25 - DNA, 24 bp, Artificial Sequence
  SYNTHESIZED
ttttttttttcattttgttttttt

SEQ ID NO: 26 - DNA, 24 bp, Artificial Sequence
  SYNTHESIZED
ttttttttttttcattttgttttt

SEQ ID NO: 27 - DNA, 24 bp, Artificial Sequence
  SYNTHESIZED
ttttttttttttttcattttgttt

SEQ ID NO: 28 - DNA, 24 bp, Artificial Sequence
  SYNTHESIZED
ttttttttttttttttcattttgt

SEQ ID NO: 29 - DNA, 24 bp, Artificial Sequence
  SYNTHESIZED
ttttcattttgtcattttgttttt

SEQ ID NO: 30 - DNA, 24 bp, Artificial Sequence
  SYNTHESIZED
tcatcaatttgtcaatttgtcatt

SEQ ID NO: 31 - DNA, 24 bp, Artificial Sequence
  SYNTHESIZED
tcatcatattgtcatattgtcatt

SEQ ID NO: 32 - DNA, 24 bp, Artificial Sequence
  SYNTHESIZED
tcatcattatgtcattatgtcatt

SEQ ID NO: 33 - DNA, 24 bp, Artificial Sequence
  SYNTHESIZED
tcatcatttagtcatttagtcatt

SEQ ID NO: 34 - DNA, 24 bp, Artificial Sequence
  SYNTHESIZED
tcatcattttatcattttatcatt

SEQ ID NO: 35 - DNA, 24 bp, Artificial Sequence
  SYNTHESIZED
tcatcattttttcattttttcatt

SEQ ID NO: 36 - DNA, 24 bp, Artificial Sequence
  SYNTHESIZED
tcatcattttctcattttctcatt

SEQ ID NO: 37 - DNA, 24 bp, Artificial Sequence
  SYNTHESIZED
tcatcattttgacattttgacatt

SEQ ID NO: 38 - DNA, 24 bp, Artificial Sequence
  SYNTHESIZED
tcatcatttagacatttagacatt

SEQ ID NO: 39 - DNA, 24 bp, Artificial Sequence
  SYNTHESIZED
tcatcattatgacattatgacatt

SEQ ID NO: 40 - DNA, 24 bp, Artificial Sequence
  SYNTHESIZED
tcatcatattgacatattgacatt

SEQ ID NO: 41 - DNA, 24 bp, Artificial Sequence
  SYNTHESIZED
tcatcattaagacattaagacatt

SEQ ID NO: 42 - DNA, 24 bp, Artificial Sequence
  SYNTHESIZED
tcatcatatagacatatagacatt

SEQ ID NO: 43 - DNA, 24 bp, Artificial Sequence
  SYNTHESIZED
tcatcataatgacataatgacatt

SEQ ID NO: 44 - DNA, 24 bp, Artificial Sequence
  SYNTHESIZED
tcatcataaagacataaagacatt

SEQ ID NO: 45 - DNA, 24 bp, Artificial Sequence
  SYNTHESIZED
tcatcaaaaagacaaaaagacatt

SEQ ID NO: 46 - DNA, 24 bp, Artificial Sequence
  SYNTHESIZED
acatcattttgtcattttgtcatt

SEQ ID NO: 47 - DNA, 24 bp, Artificial Sequence
  SYNTHESIZED
ccatcattttgtcattttgtcatt

SEQ ID NO: 48 - DNA, 24 bp, Artificial Sequence
  SYNTHESIZED
gcatcattttgtcattttgtcatt

SEQ ID NO: 49 - DNA, 24 bp, Artificial Sequence
  SYNTHESIZED
taatcattttgtcattttgtcatt

SEQ ID NO: 50 - DNA, 24 bp, Artificial Sequence
  SYNTHESIZED
ttatcattttgtcattttgtcatt

SEQ ID NO: 51 - DNA, 24 bp, Artificial Sequence
  SYNTHESIZED
tgatcattttgtcattttgtcatt

SEQ ID NO: 52 - DNA, 24 bp, Artificial Sequence
  SYNTHESIZED
tcctcattttgtcattttgtcatt

SEQ ID NO: 53 - DNA, 24 bp, Artificial Sequence
  SYNTHESIZED
tcttcattttgtcattttgtcatt

SEQ ID NO: 54 - DNA, 24 bp, Artificial Sequence
  SYNTHESIZED
tcgtcattttgtcattttgtcatt

SEQ ID NO: 55 - DNA, 24 bp, Artificial Sequence
  SYNTHESIZED
tcaacattttgtcattttgtcatt

SEQ ID NO: 56 - DNA, 24 bp, Artificial Sequence
  SEQ ID NO 56
tcaccattttgtcattttgtcatt

SEQ ID NO: 57 - DNA, 24 bp, Artificial Sequence
  SYNTHESIZED
tcagcattttgtcattttgtcatt

SEQ ID NO: 58 - DNA, 24 bp, Artificial Sequence
  SYNTHESIZED
tcatcattttgtcattttgtaatt

SEQ ID NO: 59 - DNA, 24 bp, Artificial Sequence
  SYNTHESIZED
tcatcattttgtcattttgttatt

SEQ ID NO: 60 - DNA, 24 bp, Artificial Sequence
  SYNTHESIZED
tcatcattttgtcattttgtgatt

SEQ ID NO: 61 - DNA, 24 bp, Artificial Sequence
  SYNTHESIZED
tcatcattttgtcattttgtcctt

SEQ ID NO: 62 - DNA, 24 bp, Artificial Sequence
  SYNTHESIZED
tcatcattttgtcattttgtcttt

SEQ ID NO: 63 - DNA, 24 bp, Artificial Sequence
  SYNTHESIZED
tcatcattttgtcattttgtcgtt

SEQ ID NO: 64 - DNA, 24 bp, Artificial Sequence
  SYNTHESIZED
tcatcattttgtcattttgtcaat

SEQ ID NO: 65 - DNA, 24 bp, Artificial Sequence
  SYNTHESIZED
tcatcattttgtcattttgtcact

SEQ ID NO: 66 - DNA, 24 bp, Artificial Sequence
  SYNTHESIZED
tcatcattttgtcattttgtcagt

SEQ ID NO: 67 - DNA, 24 bp, Artificial Sequence
  SYNTHESIZED
tcatcattttgtcattttgtcata

SEQ ID NO: 68 - DNA, 24 bp, Artificial Sequence
  SYNTHESIZED
tcatcattttgtcattttgtcatc

SEQ ID NO: 69 - DNA, 24 bp, Artificial Sequence
  SYNTHESIZED
tcatcattttgtcattttgtcatg

SEQ ID NO: 70 - DNA, 12 bp, Artificial Sequence
  SYNTYHESIZED
ttttcattttgt

SEQ ID NO: 71 - DNA, 16 bp, Artificial Sequence
  SYNTHESIZED
ttttcattttgttttt

SEQ ID NO: 72 - DNA, 20 bp, Artificial Sequence
  SYNTHESIZED
ttttcattttgttttttttt

SEQ ID NO: 73 - DNA, 24 bp, Artificial Sequence
  SEQ ID NO 74
ttttttttttttcattttgttttt

SEQ ID NO: 74 - DNA, 28 bp, Artificial Sequence
  SYNTHESIZED
ttttcattttgttttttttttttttttt

SEQ ID NO: 75 - DNA, 24 bp, Artificial Sequence
  SYNTHESIZED
tcatcaattggtcaattggtcatt

SEQ ID NO: 76 - DNA, 24 bp, Artificial Sequence
  SYNTHESIZED
tcatcaaatggtcaaatggtcatt

SEQ ID NO: 77 - DNA, 24 bp, Artificial Sequence
  SYNTHESIZED
tcatcaactggtcaactggtcatt

SEQ ID NO: 78 - DNA, 24 bp, Artificial Sequence
  SYNTHESIZED
tcatcaagtggtcaagtggtcatt

SEQ ID NO: 79 - DNA, 24 bp, Artificial Sequence
  SYNTHESIZED
tcatcaataggtcaataggtcatt

SEQ ID NO: 80 - DNA, 24 bp, Artificial Sequence
  SYNTHESIZED
tcatcaatcggtcaatcggtcatt

SEQ ID NO: 81 - DNA, 24 bp, Artificial Sequence
  SYNTHESIZED
tcatcaatgggtcaatgggtcatt

SEQ ID NO: 82 - DNA, 24 bp, Artificial Sequence
  SYNTHESIZED
tcatcattatgtcattatgtcatt

SEQ ID NO: 83 - DNA, 24 bp, Artificial Sequence
  SYNTHESIZED
tcatcattctgtcattctgtcatt

SEQ ID NO: 84 - DNA, 24 bp, Artificial Sequence
  SYNTHESIZED
tcatcattgtgtcattgtgtcatt

SEQ ID NO: 85 - DNA, 24 bp, Artificial Sequence
  SYNTHESIZED
tcatcatttagtcatttagtcatt

SEQ ID NO: 86 - DNA, 24 bp, Artificial Sequence
  SYNTHESIZED
tcatcatttcgtcatttcgtcatt

SEQ ID NO: 87 - DNA, 24 bp, Artificial Sequence
  SYNTHESIZED
tcatcatttggtcatttggtcatt

SEQ ID NO: 88 - DNA, 24 bp, Artificial Sequence
  SYNTHESIZED
tcatcattttgacattttgacatt

SEQ ID NO: 89 - DNA, 24 bp, Artificial Sequence
  SYNTHESIZED
tcatcattttgccattttgccatt

SEQ ID NO: 90 - DNA, 24 bp, Artificial Sequence
  SYNTHESIZED
tcatcattttggcattttggcatt

SEQ ID NO: 91 - DNA, 24 bp, Artificial Sequence
  SYNTHESIZED
tgctgcttttgtgcttttgtgctt

SEQ ID NO: 92 - DNA, 24 bp, Artificial Sequence
  SYNTHESIZED
tcatcactttggcactttggcatt

SEQ ID NO: 93 - DNA, 24 bp, Artificial Sequence
  SYNTHESIZED
tcatcagtttggcagtttggcatt

SEQ ID NO: 94 - DNA, 24 bp, Artificial Sequence
  SYNTHESIZED
tcatcatattggcatattggcatt

SEQ ID NO: 95 - DNA, 24 bp, Artificial Sequence
  SYNTHESIZED
tcatcatcttggcatcttggcatt

SEQ ID NO: 96 - DNA, 24 bp, Artificial Sequence
  SYNTHESIZED
tcatcatgttggcatgttggcatt

SEQ ID NO: 97 - DNA, 24 bp, Artificial Sequence
  SYNTHESIZED
tgctgcccccgcgcccccgcgccc

SEQ ID NO: 98 - DNA, 24 bp, Artificial Sequence
  SYNTHESIZED
ggggtttttttttttttttttttt

SEQ ID NO: 99 - DNA, 24 bp, Artificial Sequence
  SYNTHESIZED
ggggtctttttttctttttttctt

SEQ ID NO: 100 - DNA, 24 bp, Artificial Sequence
  SYNTHESIZED
tgctgcccccgcgcccccgcgccc

SEQ ID NO: 101 - DNA, 24 bp, Artificial Sequence
  SYNTHESIZED
ggggacttttatacttttatactt

SEQ ID NO: 102 - DNA, 24 bp, Artificial Sequence
  SYNTHESIZED
ggggtatttttttatttttttatt

SEQ ID NO: 103 - DNA, 24 bp, Artificial Sequence
  SYNTEHSIZED
gggggcttttgggcttttgggcgg

SEQ ID NO: 104 - DNA, 24 bp, Artificial Sequence
  SYNTHESIZED
gggggctttagtgctttagtgctt

SEQ ID NO: 105 - DNA, 24 bp, Artificial Sequence
  SYNTHESIZED
gggggctaatgtgctaatgtgctt

SEQ ID NO: 106 - DNA, 24 bp, Artificial Sequence
  SYNTHESIZED
tcgtcgaaaagtcgaaaagtcgaa

SEQ ID NO: 107 - DNA, 24 bp, Artificial Sequence
  SYNTHESIZED
gggggtctttttttcttttttttt

SEQ ID NO: 108 - DNA, 24 bp, Artificial Sequence
  SYNTHESIZED
ttttttctttttttcttttttggg

SEQ ID NO: 109 - DNA, 24 bp, Artificial Sequence
  SYNTHESIZED
aaaaatctttttttcttttttttt

SEQ ID NO: 110 - DNA, 24 bp, Artificial Sequence
  SYNTHESIZED
tgctgcttttatgcttttatgctt

SEQ ID NO: 111 - DNA, 24 bp, Artificial Sequence
  SYNTHESIZED
tcatcattctgtcattctgtcatt

SEQ ID NO: 112 - DNA, 24 bp, Artificial Sequence
  SYNTHESIZED
ttttttctttttttcttttttttt

SEQ ID NO: 113 - DNA, 24 bp, Artificial Sequence
  SYNTHESIZED
tgctgcttttctgcttttctgctt

SEQ ID NO: 114 - DNA, 24 bp, Artificial Sequence
  SYNTHESIZED
ttttttcttttctttttttttttt

SEQ ID NO: 115 - DNA, 24 bp, Artificial Sequence
  SYNTHESIZED
tttttccttttttccttttttttt

SEQ ID NO: 116 - DNA, 24 bp, Artificial Sequence
  SYNTHESIZED
ccccctctttttttcttttttttt

SEQ ID NO: 117 - DNA, 24 bp, Artificial Sequence
  SYNTHESIZED
ttttttctttttcttttttttttt

SEQ ID NO: 118 - DNA, 24 bp, Artificial Sequence
  SYNTHESIZED
ttttttctttttttcttttttccc

SEQ ID NO: 119 - DNA, 24 bp, Artificial Sequence
  SYNTHESIZED
ttttttctttttctctttttctct

SEQ ID NO: 120 - DNA, 24 bp, Artificial Sequence
  SYNTHESIZED
tttttgcttttttgcttttttttt

SEQ ID NO: 121 - DNA, 24 bp, Artificial Sequence
  SYNTHESIZED′
tttttacttttttacttttttttt

SEQ ID NO: 122 - DNA, 23 bp, Artificial Sequence
  SYNTHESIZED
cataattttgtaattttgtcatt

SEQ ID NO: 123 - DNA, 24 bp, Artificial Sequence
  SYNTHESIZED
ttttttctttttttcttttttaaa

SEQ ID NO: 124 - DNA, 24 bp, Artificial Sequence
  SYNTHESIZED
ttttttcttttttctttttttttt

SEQ ID NO: 125 - DNA, 24 bp, Artificial Sequence
  SYNTHESIZED
ttttttttttttcattttgtgggg

SEQ ID NO: 126 - DNA, 24 bp, Artificial Sequence
  SYNTHESIZED
ttttttttttttcattttgttttg

SEQ ID NO: 127 - DNA, 24 bp, Artificial Sequence
  SYNTHESIZED
gggtttttttttcattttgttttt

SEQ ID NO: 128 - DNA, 24 bp, Artificial Sequence
  SYNTHESIZED
gtttttttttttcattttgttttg

(19) United States
(12) Patent Application Publication
(10) Pub. No.: US 2008/0107706 A1
(43) Pub. Date: May 8, 2008

(54) Osteogenic Oligonucleotides and Uses Thereof

(76) Inventor: Ricardo Agustin Lopez, Buenos Aires (AR)

Correspondence Address: JACOBSON HOLMAN PLLC, 400 SEVENTH STREET N.W., SUITE 600, WASHINGTON, DC, 20004, US

(21) Appl. No.: 11/663,833

(22) PCT Filed: Sep. 12, 2005
(86) PCT No.: PCT/EP05/54521

§ 371 Date: Mar. 27, 2007

(30) Foreign Application Priority Data
Sep. 27, 2004 (EP) 04077653.6

(51) Int. Cl. A61F 2/00; A61K 31/70

(52) U.S. Cl. 424/423; 514/44

(57) ABSTRACT

Oligonucleotides having the ability to stimulate osteogenesis in vertebrate animals, including humans, are disclosed. These osteogenic oligonucleotides can be used in a wide range of clinical procedures to replace and restore osseous and periodontal defects or to facilitate the successful implantation of prosthesis and distraction procedures in bones. Methods for generation of bone in an area of an animal where skeletal tissue is deficient are provided. They consist in the local or systematic administration of a composition comprising one or more of the osteogenic oligonucleotides to the animal, in a pharmaceutically acceptable carrier. The composition is administered in an amount effective to induce osteogenesis at the bone site.

### FIELD OF THE INVENTION

This invention relates to the use of both osteogenic oligonucleotides and pharmaceutical compositions to induce bone growth in vivo. More in details it refers to oligonucleotides having about 14 to 100 nucleotides that have the ability to stimulate the osteogenesis in animals, including human.

### REFERENCES

### U.S. Patent Documents

1) U.S. Pat. No. 5,409,896 Apr. 25, 1995
2) U.S. Pat. No. 5,422,340 Jun. 6, 1995
3) U.S. Pat. No. 5,604,204 Feb. 18, 1997
4) U.S. Pat. No. 5,854,207 Dec. 29, 1998
5) U.S. Pat. No. 5,885,964 Mar. 23, 1999
6) U.S. Pat. No. 5,948,428 Sep. 7, 1999
7) U.S. Pat. No. 5,972,703 Oct. 26, 1999
8) U.S. Pat. No. 6,028,207 Feb. 22, 2000
9) U.S. Pat. No. 6,355,672 Mar. 12, 2002
10) U.S. Pat. No. 6,426,222 Jul. 30, 2002
11) U.S. Pat. No. 6,531,604 Mar. 11, 2003
12) U.S. Pat. No. 6,569,204 May 27, 2003
13) U.S. Pat. No. 6,649,072 Nov. 18, 2003

### Non U.S. Patent Documents

1) WO 98/08517 Mar. 5, 1998
2) WO 98/30234 Jul. 16, 1998
3) WO 98/33515 Aug. 6, 1998
4) WO 00/78351 Dec. 28, 2000
5) WO 01/10463 Feb. 15, 2001
6) WO 01/89521 Nov. 29, 2001
7) WO 02/080955 Oct. 17, 2002
8) WO 2004/030683 Apr. 15, 2004

### Other References

1) Hughes F J, McCulloch C A. Stimulation of the differentiation of osteogenic rat bone marrow stromal cells by osteoblast cultures. Lab Invest. 1991 May; 64(5):617-22.
2) Lee S C, Shea M, Battle M A, Kozitza K, Ron E, Turek T, Schaub R G, Hayes W C. Healing of large segmental defects in rat femurs is aided by RhBMP-2 in PLGA matrix. J Biomed Mater Res. 1994 October; 28(10): 1149-56.
3) Asahina I, Sampath T K, Hauschka P V. Human osteogenic protein-1 induces chondroblastic, osteoblastic, and/or adipocytic differentiation of clonal murine target cells. Exp Cell Res. 1996 Jan. 10; 222(1):38-47.
4) Maliakal J C, Asahina I, Hauschka P V, Sampath T K. Osteogenic protein-1 (BMP-7) inhibits cell proliferation and stimulates the expression of markers characteristic of osteoblast phenotype in rat osteosarcoma (17/2.8) cells. Growth Factors. 1994; 11(3):227-34.
5) Asahina I, Sampath T K, Nishimura I, Hauschka P V. Human osteogenic protein-1 induces both chondroblastic and osteoblastic differentiation of osteoprogenitor cells derived from newborn rat calvaria. J. Cell Biol. 1993 November; 123(4):921-33.
6) Akiyama H, Fukumoto A, Shigeno C, Ito H, Mukai S, Hoshino T, Makino H, Nakamura T. TAK-778, a novel synthetic 3-benzothiepin derivative, promotes chondrogenesis in vitro and in vivo. Biochem Biophys Res Commun. 1999 Jul. 22; 261(1):131-8.
7) Notoya K, Nagai H, Oda T, Gotoh M, Hoshino T, Muranishi H, Taketomi S, Sohda T, Makino H. Enhancement of osteogenesis in vitro and in vivo by a novel osteoblast differentiation promoting compound, TAK-778. J Pharmacol Exp Ther. 1999 September; 290(3):1054-64.
8) Oda T, Notoya K, Gotoh M, Taketomi S, Fujisawa Y, Makino H, Sohda T. Synthesis of novel 2-benzothiopyran and 3-benzothiepin derivatives and their stimulatory effect on bone formation. J Med. Chem. 1999 Feb. 25; 42(4):751-60.
9) Sigurdsson T J, Nygaard L, Tatakis D N, Fu E, Turek T J, Jin L, Wozney J M, Wikesjo U M. Periodontal repair in dogs: evaluation of rhBMP-2 carriers. Int J Periodontics Restorative Dent. 1996 December; 16(6):524-37.
10) Hanada K, Dennis J E, Caplan A I. Stimulatory effects of basic fibroblast growth factor and bone morphogenetic protein-2 on osteogenic differentiation of rat bone marrow-derived mesenchymal stem cells. J Bone Miner Res. 1997 October; 12(10):1606-14.
11) King G N, King N, Cruchley A T, Wozney J M, Hughes F J. Recombinant human bone morphogenetic protein-2 promotes wound healing in rat periodontal fenestration defects. J Dent Res. 1997 August; 76(8):1460-70.
12) Kimura M, Zhao M, Zellin G, Linde A. Bone-inductive efficacy of recombinant human bone morphogenetic protein-2 expressed in Escherichia coli: an experimental study in rat mandibular defects. Scand J Plast Reconstr Surg Hand Surg. 2000 December; 34(4):289-99.
13) Zellin G, Linde A. Effects of recombinant human fibroblast growth factor-2 on osteogenic cell populations during orthopic osteogenesis in vivo. Bone. 2000 February; 26(2):161-8.
14) Zellin G, Linde A. Treatment of segmental defects in long bones using osteopromotive membranes and recombinant human bone morphogenetic protein-2. An experimental study in rabbits. Scand J Plast Reconstr Surg Hand Surg. 1997 June; 31(2):97-104.
15) Zellin G, Linde A. Importance of delivery systems for growth-stimulatory factors in combination with osteopromotive membranes. An experimental study using rhBMP-2 in rat mandibular defects. J Biomed Mater Res. 1997 May; 35(2):181-90.
16) Wikesjo U M, Guglielmoni P, Promsudthi A, Cho K S, Trombelli L, Selvig K A, Jin L, Wozney J M. Periodontal repair in dogs: effect of rhBMP-2 concentration on regeneration of alveolar bone and periodontal attachment. J Clin Periodontol. 1999 June; 26(6):392-400.
17) Barboza E, Caula A, Machado F. Potential of recombinant human bone morphogenetic protein-2 in bone regeneration. Implant Dent. 1999; 8(4):360-7. Review.
18) Murata M, Huang B Z, Shibata T, Imai S, Nagai N, Arisue M. Bone augmentation by recombinant human BMP-2 and collagen on adult rat parietal bone. Int J Oral Maxillofac Surg. 1999 June; 28(3):232-7.
19) Cochran D L, Schenk R, Buser D, Wozney J M, Jones A A. Recombinant human bone morphogenetic protein-2 stimulation of bone formation around endosseous dental implants. J Periodontol. 1999 February; 70(2):139-50.
20) Isobe M, Yamazaki Y, Mori M, Ishihara K, Nakabayashi N, Amagasa T. The role of recombinant human bone morphogenetic protein-2 in PLGA capsules at an extraskeletal site of the rat. J Biomed Mater Res. 1999 April; 45(1):36-41.
21) Mori M, Isobe M, Yamazaki Y, Ishihara K, Nakabayashi N. Restoration of segmental bone defects in rabbit radius by biodegradable capsules containing recombinant human bone morphogenetic protein-2. J Biomed Mater Res. 2000 May; 50(2):191-8.
22) Hoshino T, Saito K, Muranishi H, Sohda T, Ogawa Y. Sustained-release microcapsules of a bone formation stimulant, TAK-778, for local injection into a fracture site. J Pharm Sci. 2001 December; 90(12):2121-30.
23) Hoshino T, Muranishi H, Saito K, Notoya K, Makino H, Nagai H, Sohda T, Ogawa Y. Enhancement of fracture repair in rats with streptozotocin-induced diabetes by a single injection of biodegradable microcapsules containing a bone formation stimulant, TAK-778. J Biomed Mater Res. 2000 Sep. 5; 51(3):299-306.
24) Kato H, Nishiguchi S, Furukawa T, Neo M, Kawanabe K, Saito K, Nakamura T. Bone bonding in sintered hydroxyapatite combined with a new synthesized agent, TAK-778. J Biomed Mater Res. 2001 Mar. 15; 54(4):619-29.
25) Yudell R M, Block M S. Bone gap healing in the dog using recombinant human bone morphogenetic protein-2. J Oral Maxillofac Surg. 2000 July; 58(7):761-6.
26) Tatakis D N, Wikesjo U M, Razi S S, Sigurdsson T J, Lee M B, Nguyen T, Ongpipattanakul B, Hardwick R. Periodontal repair in dogs: effect of transforming growth factor-beta 1 on alveolar bone and cementum regeneration. J Clin Periodontol. 2000 September; 27(9):698-704.
27) Danesh-Meyer M J. Tissue engineering in periodontics and implantology using rhBMP-2. Ann R Australas Coll Dent Surg. 2000 October; 15:144-9.
28) Oldham J B, Lu L, Zhu X, Porter B D, Hefferan T E, Larson D R, Currier B L, Mikos A G, Yaszemski M J. Biological activity of rhBMP-2 released from PLGA microspheres. J Biomech Eng. 2000 June; 122(3):289-92.
29) Marukawa E, Asahina I, Oda M, Seto I, Alam M I, Enomoto S. Bone regeneration using recombinant human bone morphogenetic protein-2 (rhBMP-2) in alveolar defects of primate mandibles. Br J Oral Maxillofac Surg. 2001 December; 39(6):452-9.
30) Seto I, Asahina I, Oda M, Enomoto S. Reconstruction of the primate mandible with a combination graft of recombinant human bone morphogenetic protein-2 and bone marrow. J Oral Maxillofac Surg. 2001 January; 59(1):53-61
31) Sykaras N, Triplett R G, Nunn M E, Iacopino A M, Opperman L A. Effect of recombinant human bone morphogenetic protein-2 on bone regeneration and osseointegration of dental implants. Clin Oral Implants Res. 2001 August; 12(4):339-49.
32) Wikesjo U M, Sorensen R G, Wozney J M. Augmentation of alveolar bone and dental implant osseointegration: clinical implications of studies with rhBMP-2. J Bone Joint Surg Am. 2001; 83-A Suppl 1(Pt 2):S136-45.
33) Majumdar M K, Wang E, Morris E A. BMP-2 and BMP-9 promotes chondrogenic differentiation of human multipotential mesenchymal cells and overcomes the inhibitory effect of IL-1. J Cell Physiol. 2001 December; 189(3):275-84.
34) Takayama S, Murakami S, Shimabukuro Y, Kitamura M, Okada H. Periodontal regeneration by FGF-2 (bFGF) in primate models. J Dent Res. 2001 December; 80(12):2075-9.
35) Ripamonti U, Crooks J, Petit J C, Rueger D C. Periodontal tissue regeneration by combined applications of recombinant human osteogenic protein-1 and bone morphogenetic protein-2. A pilot study in Chacma baboons (Papio ursinus). Eur J Oral Sci. 2001 August; 109(4):241-8.
36) Hoshino T, Saito K, Muranishi H, Sohda T, Ogawa Y. Sustained-release microcapsules of a bone formation stimulant, TAK-778, forlocal injection into a fracture site. J Pharm Sci. 2001 December; 90(12):2121-30.
37) Kato H, Neo M, Tamura J, Nakamura T. Bone bonding in bioactive glass ceramics combined with a new synthesized agent TAK-778. J Biomed Mater Res. 2001 November; 57(2):291-9.
38) Kato H, Nishiguchi S, Furukawa T, Neo M, Kawanabe K, Saito K, Nakamura T. Bone bonding in sintered hydroxyapatite combined with a new synthesized agent, TAK-778. J Biomed Mater Res. 2001 Mar. 15; 54(4):619-29.
39) Gotoh M, Notoya K, Ienaga Y, Kawase M, Makino H. Enhancement of osteogenesis in vitro by a novel osteoblast differentiation-promoting compound, TAK-778, partly through the expression of Msx2. Eur J Pharmacol. 2002 Sep. 6; 451(1):19-25.
40) Anusaksathien O, Giannobile W V. Growth factor delivery to re-engineer periodontal tissues. Curr Pharm Biotechnol. 2002 June; 3(2):129-39.
41) Lieberman J R, Daluiski A, Einhorn T A. The role of growth factors in the repair of bone. Biology and clinical applications. J Bone Joint Surg Am. 2002 June; 84-A(6):1032-44.
42) Saltzman W M, Olbricht W L. Building drug delivery into tissue engineering. Nat Rev Drug Discov. 2002 March; 1(3):177-86.
43) Li G, Bouxsein M L, Luppen C, Li X J, Wood M, Seeherman H J, Wozney J M, Simpson H. Bone consolidation is enhanced by rhBMP-2 in a rabbit model of distraction osteogenesis. J Orthop Res. 2002 July; 20(4):779-88.
44) Weber F E, Eyrich G, Gratz K W, Maly F E, Sailer H F. Slow and continuous application of human recombinant bone morphogenetic protein via biodegradable poly(lactide-co-glycolide) foamspheres. Int J Oral Maxillofac Surg. 2002 February; 31(1):60-5.
45) King G N, Cochran D L. Factors that modulate the effects of bone morphogenetic protein-induced periodontal regeneration: a critical review. J Periodontol. 2002 August; 73(8):925-36.
46) Schilephake H. Bone growth factors in maxillofacial skeletal reconstruction. Int J Oral Maxillofac Surg. 2002 October; 31(5):469-84.
47) Ikeuchi M, Dohi Y, Horiuchi K, Ohgushi H, Noshi T, Yoshikawa T, Yamamoto K, Sugimura M. Recombinant human bone morphogenetic protein-2 promotes osteogenesis within atelopeptide type I collagen solution by combination with rat cultured marrowcells. J Biomed Mater Res. 2002 April; 60(1):61-9.
48) Marukawa E, Asahina I, Oda M, Seto I, Alam M, Enomoto S. Functional reconstruction of the non-human primate mandible using recombinant human bone morphogenetic protein-2. Int J Oral Maxillofac Surg. 2002 June; 31(3):287-95.
49) Selvig K A, Sorensen R G, Wozney J M, Wikesjo U M. Bone repair following recombinant human bone morphogenetic protein-2 stimulated periodontal regeneration. J Periodontol. 2002 September; 73(9):1020-9.
50) Rosa A L, Beloti M M. TAK-778 enhances osteoblast differentiation of human bone marrow cells. J Cell Biochem. 2003 Aug. 15; 89(6):1148-53.
51) Cook S D, Barrack R L, Patron L P, Salkeld S L. Osteoinductive agents in reconstructive hip surgery: a look forward. Clin Orthop. 2003 December; (417):195-202.
52) Puleo D. Biotherapeutics in orthopaedic medicine: accelerating the healing process? BioDrugs. 2003; 17(5):301-14.
53) Tabata Y. Tissue regeneration based on growth factor release. Tissue Eng. 2003; 9 Suppl 1:S5-15.
54) Wikesjo U M, Xiropaidis A V, Thomson R C, Cook A D, Selvig K A, Hardwick W R. Periodontal repair in dogs: rhBMP-2 significantly enhances bone formation under provisions for guided tissue regeneration. J Clin Periodontol. 2003 August; 30(8):705-14.
55) Matin K, Senpuku H, Hanada N, Ozawa H, Ejiri S. Bone regeneration by recombinant human bone morphogenetic protein-2 around immediate implants: a pilot study in rats. Int J Oral Maxillofac Implants. 2003 March-April; 18(2):211-7.
56) Nakashima M, Reddi A H. The application of bone morphogenetic proteins to dental tissue engineering. Nat. Biotechnol. 2003 September; 21(9):1025-32.
57) Camelo M, Nevins M L, Schenk R K, Lynch S E, Nevins M. Periodontal regeneration in human Class II furcations using purified recombinant human platelet-derived growth factor-BB (rhPDGF-BB) with bone allograft. Int J Periodontics Restorative Dent. 2003 June; 23(3):213-25.
58) Sheehan J P, Sheehan J M, Seeherman H, Quigg M, Helm G A. The safety and utility of recombinant human bone morphogenetic protein-2 for cranial procedures in a nonhuman primate model. J. Neurosurg. 2003 January; 98(1):125-30.
59) Murakami S, Takayama S, Kitamura M, Shimabukuro Y, Yanagi K, Ikezawa K, Saho T, Nozaki T, Okada H. Recombinant human basic fibroblast growth factor (bFGF) stimulates periodontal regeneration in class II furcation defects created in beagle dogs. J Periodontal Res. 2003 February; 38(1):97-103.
60) Rasubala L, Yoshikawa H, Nagata K, Iijima T, Ohishi M. Platelet-derived growth factor and bone morphogenetic protein in the healing of mandibular fractures in rats. Br J Oral Maxillofac Surg. 2003 June; 41(3):173-8.
61) Clokie C M, Bell R C. Recombinant human transforming growth factor beta-1 and its effects on osseointegration. J Craniofac Surg. 2003 May; 14(3):268-77.
62) Lee Y M, Nam S H, Seol Y J, Kim T I, Lee S J, Ku Y, Rhyu I C, Chung C P, Han S B, Choi S M. Enhanced bone augmentation by controlled release of recombinant human bone morphogenetic protein-2 from bioabsorbable membranes. J Periodontol. 2003 June; 74(6):865-72.
63) Sykaras N, Opperman L A. Bone morphogenetic proteins (BMPs): how do they function and what can they offer the clinician? J Oral Sci. 2003 June; 45(2):57-73.
64) Sykaras N, Opperman L A. Bone morphogenetic proteins (BMPs): how do they function and what can they offer the clinician? J Oral Sci. 2003 June; 45(2):57-73.
65) Schmidmaier G, Wildemann B, Gabelein T, Heeger J, Kandziora F, Haas N P, Raschke M. Synergistic effect of IGF-I and TGF-beta1 on fracture healing in rats: single versus combined application of IGF-I and TGF-beta1. Acta Orthop Scand. 2003 October; 74(5):604-10.
66) Cheng H, Jiang W, Phillips F M, Haydon R C, Peng Y, Zhou L, Luu H H, An N, Breyer B, Vanichakam P, Szatkowski J P, Park J Y, He T C. Osteogenic activity of the fourteen types of human bone morphogenetic proteins (BMPs). J Bone Joint Surg Am. 2003 August; 85-A(8):1544-52.
67) van den Dolder J, de Ruijter A J, Spauwen P H, Jansen J A. Observations on the effect of BMP-2 on rat bone marrow cells cultured on titanium substrates of different roughness. Biomaterials. 2003 May; 24(11):1853-60.
68) Matin K, Senpuku H, Hanada N, Ozawa H, Ejiri S. Bone regeneration by recombinant human bone morphogenetic protein-2 around immediate implants: a pilot study in rats. Int J Oral Maxillofac Implants. 2003 March-April; 18(2):211-7.
69) Wikesjo U M, Qahash M, Thomson R C, Cook A D, Rohrer M D, Wozney J M, Hardwick W R. Space-providing expanded polytetrafluoroethylene devices define alveolar augmentation at dental implants induced by recombinant human bone morphogenetic protein 2 in an absorbable collagen sponge carrier. Clin Implant Dent Relat Res. 2003; 5(2):112-23.
70) Wikesjo U M, Qahash M, Thomson R C, Cook A D, Rohrer M D, Wozney J M, Hardwick W R. rhBMP-2 significantly enhances guided bone regeneration. Clin Oral Implants Res. 2004 April; 15(2):194-204.
71) Benghuzzi H, Tucci M, Tsao A, Russell G, England B, Ragab A. Stimulation of osteogenesis by means of sustained delivery of various natural androgenic hormones. Biomed Sci Instrum. 2004; 40:99-104.
72) Rosa A L, Beloti M M. TAK-778 enhances osteoblast differentiation of human bone marrow cells via an estrogen-receptor-dependent pathway. J Cell Biochem. 2004 Mar. 1; 91(4):749-55.
73) Roldan J C, Jepsen S, Miller J, Freitag S, Rueger D C, Acil Y, Terheyden H. Bone formation in the presence of platelet-rich plasma vs. bone morphogenetic protein-7. Bone. 2004 January; 34(1):80-90.
74) Ashinoff R L, Cetrulo C L Jr, Galiano R D, Dobryansky M, Bhatt K A, Ceradini D J, Michaels J 5th, McCarthy J G, Gurtner G C. Bone morphogenic protein-2 gene therapy for mandibular distraction osteogenesis. Ann Plast Surg. 2004 June; 52(6):585-90.
75) Osyczka A M, Diefenderfer D L, Bhargave G, Leboy P S. Different effects of BMP-2 on marrow stromal cells from human and rat bone. Cells Tissues Organs. 2004; 176(1-3):109-19.

### BACKGROUND OF THE INVENTION

Bone is a complex, highly organized, connective tissue that is continuously remodelled during the life of an adult by cellular events that initially break it down (osteoclastic resorption) and then rebuild it (osteoblastic formation). This remodelling process occurs in discrete sites throughout the skeleton. Bone is the only organ capable of complete repair without the intervention of a fibrous scar (Hult. A. 1989. Current concepts of fracture healing. Clin. Orthop. Relat. Res. 249:265-384.). However, there are clinical situations that require enhancement of the healing to ensure the rapid restoration of bone function, such as orthopaedic and maxillofacial surgery. On the other hand, some events, including aging, poor blood supply, and diabetes, may lead to prevent fracture healing (J. A. Buckwalter, T. A. Einhorn, M. E. Bolander and R. L. Cruess, Healing of the musculoskeletal tissues. In: C. A. Rockwood, D. P. Green, R. W. Bucholz and J. D. Heckman, Editors, Fracture in Adults, Lippincott-Raven, New York (1996), pp. 261-304.). The restoration of an osteoporotic fracture is also delayed since the mechanical strength of the fracture site is decreased due to an insufficient amount of callus and calcification (L. J. Melton, III, Epidemiology of fractures. In: B. L. Riggs and L. J. Melton, III, Editors, Osteoporosis: Etiology, Diagnosis, and Management, Lippincott-Raven, Philadelphia (1995), pp. 225-247; W. R. Walsh, P. Sherman, C. R. Howlett, D. H. Sonnabend and M. G. Ehrlich, Fracture healing in a rat osteopenia model. Clin. Orthop. Relat. Res. 342 (1997), pp. 213-227.). Thus, the development of agents to enhance osteogenesis would be a significant pharmacological advancement in terms of accelerating both fracture healing and the surgical procedures involving bone repair and of preventing fractures in patients suffering osteogenic dysfunctions.

A number of growth factors and cytokines are present in high levels at fracture sites and many of these proteins play important roles in promoting bone repair (M. E. Bolander, Regulation of fracture repairs by growth factors. Proc. Soc. Exp. Biol. Med. 200 (1992), pp. 165-170.). Drug therapies using various growth factors such as bone morphogenetic proteins (BMPs), transforming growth factor β (TGF-β), insulin-like growth factors (IGFs), fibroblast growth factors (FGFs), and platelet-derived growth factor (PDGF) may be useful since local application of these molecules has been shown to induce bone regeneration in animal models (Lind M (1996) Growth factors: Possible new clinical tools. Acta Orthop. Scand. 67: 407-417).

These findings indicate that the clinical use of these growth factors may become possible new therapies for enhancing fracture healing. However, the safety and cost-effectiveness of these growth factors must be considered. Therefore, there has been substantial interest in developing chemical compounds that safely promote bone formation and facilitate fracture repair. One example of such a compound is the TAK-778, a derivative of the ipriflavone (7-isopropoxy-isoflavone) which has osteogenic activity “in vitro” and “in vivo” (: Notoya K, Nagai H, Oda T, Gotoh M, Hoshino T, Muranishi H, Taketomi S, Sohda T, Makino H (1999). Enhancement of osteogenesis in vitro and in vivo by a novel osteoblast differentiation promoting compound, TAK-778. J Pharmacol Exp Ther. 290:1054-64). Utility of this compound for human use must be proved in clinical trials.

We now disclose that oligonucleotides having about 14 to 100 nucleotides are compounds with potent osteogenic activity.

It is an object of the present invention to provide one or more of the osteogenic oligonucleotides of this invention to an animal with a skeletal (bony) tissue deficiency to produce mature, morphologically normal bone where it is needed. This object will become apparent to those skilled in the art.

### SUMMARY OF THE INVENTION

The above object is achieved by providing a method for bone generation at a site of an animal where skeletal tissue is deficient and which consists in the administration of an effective amount of a composition comprising one or more of the osteogenic oligonucleotides of this invention to the animal, locally at the site or systemically as needed in each case, in a pharmaceutically acceptable carrier, the composition being administered in an amount effective to induce bone growth at the site.

This aspect of the invention enables the generation of normal mature bone in the skeleton in general or locally as required. Pre-clinical results using as example some of the osteogenic ODNs of this invention described below show new bone formation in bone defects in rats, and new bone formation in bone defects in primates.

### BRIEF DESCRIPTION OF THE DRAWINGS

FIG. 1 shows an X-ray radiographic analysis of the tibias of a rat, at osteotomy sites, 7, 21 and 35 days after operation, both in the placebo (A, B and C) and in IMT504-treated (D, E and F) bone.

FIG. 2 shows photographs of the tibias of a rat, at osteotomy sites, 35 days after operation both in the placebo (A) and in IMT504-treated (B) bone.

FIG. 3 shows microphotographs of the tibias of a rat, at osteotomy sites, 35 days after operation both in the placebo (A) and in IMT504-treated (B) bone.

### DESCRIPTION OF THE PREFERRED EMBODIMENTS

“Inducing bone growth” means promoting the formation of morphologically normal, mature bone only at a site where there is a bone deficiency that needs to be replaced. Mature bone is bone of any type, whether cortical or trabecular, that is mineralized as opposed to immature or cartilaginous bone as would be formed in a neonatal model. Morphologically normal bone is bone that is histologically detected as normal (i.e., consisting of endochondral or membranous type lamellar bone and including marrow spaces with osteoblasts and osteoclasts). This is in contrast, for example, to callous formation with a fibrotic matrix as seen in the first stage of fracture healing. Thus, the bone induction herein is contemplated not only as acceleration of bone regeneration, as in a fracture, but also as stimulation of the formation of bone that is returned to its normal morphological state. “Skeletal tissue deficiency” refers to a deficiency in bone at any site, originated as a result of either surgical intervention or fracture, and where bone it is desired to restore the bone.

By “osteogenesis” is meant the process by which bone develops.

By “osteogenic” cells is meant cells able to proliferate and to differentiate into osteoblasts and osteocytes.

By “osteoblasts” is meant mature bone cell concerned with synthesis and secretion of bone extracellular organic constituents.

By “osteocytes” is meant cells that are essentially osteoblasts surrounded by the products they secrete.

By “animal” is meant any animal having a vertebrate structure, preferably a mammal, and most preferably a human.

A “subject” refers to an animal of the order primate, including humans.

As used herein, the term “oligonucleotide” or “oligo” shall mean multiple nucleotides (i.e. molecules comprising a sugar, e.g. ribose or deoxyribose, linked to a phosphate group and to an exchangeable organic base, which is either a substituted pyrimidine (e.g. cytosine (C), thymine (T) or uracil (U)) or a substituted purine (e.g. adenine (A) or guanine (G)). The term “oligonucleotide” as used herein refers to both oligoribonucleotides (ORNs) and oligodeoxyribonucleotides (ODNs). The term “oligonucleotide” shall also include oligonucleosides (i.e. an oligonucleotide minus the phosphate) and any other organic base containing polymer. Oligonucleotides can be obtained from existing nucleic acid sources (e.g. genomic or cDNA), but are preferably synthetic (e.g. produced by oligonucleotide synthesis).

An “oligonucleotide” refers to multiple nucleotides linked by phosphodiester bonds.

An “immunostimulatory oligonucleotide” refers to an oligonucleotide which stimulates (i.e. has a mitogenic effect on, induces, increases or decreases cytokine expression by) a cell of the immune system (i.e. a lymphocyte or a macrophage) in a statistically significant manner.

A “CpG” refers to a cytosine-guanine dinucleotide.

A “CpG oligonucleotide” refers to an oligonucleotide which stimulates a cell of the immune system, and whose immunostimulatory activity critically depends on the presence of at least one CpG in its sequence.

A “non-CpG oligonucleotide” refers to an oligonucleotide that stimulates a cell of the immune system, and whose immunostimulatory activity does not critically depend on the presence of a CpG in its sequence.

### MODES FOR CARRYING OUT THE INVENTION

The invention is carried out in one aspect by mixing one or more of the osteogenic oligonucleotides of this invention with a suitable pharmaceutical carrier and by administering the resulting composition locally or systemically as required to an animal in order to induce formation of normal, adult bone in bone lesions. Osteogenic cells and their precursor cells should be present at the lesion site or sites. If the lesion site or sites does not naturally have a source of osteogenic cells present, the pharmaceutical composition may also contain an osteogenic cell source, in an amount sufficient to induce bone growth.

Examples of indications where promotion of bone repair at a skeletal site or sites is important include: periodontal disease where root socket healing is impaired (tooth socket sites), non-union fractures (including primary treatment of high risk fractures and adjunctive treatment with bone grafting or bone substitutes for established non-union fractures), large bony defects caused by trauma or surgery [e.g., partial mandibular resection for cancer, large cranial defects, spinal (vertebral) fusions, correction of severe scoliosis by surgical alignment held in place with a Harrington bar (to shorten the six-month period normally required for a body cast), and spinal fractures with open reduction (to decrease significantly the period of immobilization)], and rapid stabilization and enhanced fixation of artificial prostheses and spacer bars, oral joints, and bone replacements.

Examples of the latter include plastic and reconstructive surgery, fixation of permanent dentures into mandible, enhanced fixation of accepted Joint prosthesis, e.g., hips, knees, and shoulders (leading to the acceptance of prostheses that are unacceptable due to rapid loosening and instability), and limb salvage procedures, usually associated with malignancy (the bone shaft may be removed but the articular surfaces are left in place and connected by a space bar; rapid and enhanced fixation is required for success). If the site constitutes a periodontal site, i.e., one that involves the teeth, gums, and dental sockets, the osteogenic oligonucleotides of this invention could be administered in conjunction with an exogenously added source of osteogenic cells.

In one preferred embodiment, the osteogenic oligonucleotides of this invention are administered by coating a device with the composition containing one or more of the oligonucleotides of this invention and by implanting the device into the animal at the site of the deficiency. The composition may also contain an osteogenic cell source when the site is deficient in such cells. The device may consist in any device suitable for implantation, including a molded implant, plug, prosthetic device, capsule, titanium alloy, sponge, or ceramic block. Examples of suitable delivery vehicles useful as devices are those disclosed by Nade et al., Clin. Orthop. Rel. Res., 181: 255-263 (1982); Uchida et al., J. Biomed. Mat. Res., 21: 1-10 (1987); Friedenstein et al., Exp. Hematol., 10: 217-227 (1982); Deporter et al., Calcif. Tissue Int., 42: 321-325 (1988); McDavid et al., J. Dent. Res., 58: 478-483 (1979); Ohgushi et al., J. Orthopaedic Res., 7: 568-578 (1989), Aprahamian et al., J. Biomed. Mat. Res., 21: 965-977 (1986) and Emmanual et al., Stain. Tech., 62: 401-409 (1987).

For bone defects involving gaps, such as a dry socket or a non-union fracture, a plug may be used to fill the gap. The plug may be composed of, for example, hydroxyapatite or collagen on which the composition containing one or more of the oligonucleotides of this invention adsorbed. For larger bone defects resulting from, e.g., trauma or skeletal reconstruction around an ulcer or hip prosthesis, the device is preferably a made-to-fit ceramic block. More preferably, the ceramic block comprises 0-100% hydroxyapatite and the remaining 100-0% tricalcium phosphate, by weight, most preferably 60% hydroxyapatite and 40% tricalcium phosphate.

In a specific embodiment for a jaw implant, a calcium carbonate moldable material or Interpore™ molding device is molded to fit the jaw, using a 3-dimensional x-ray of the jaw before surgery. The molded material is impregnated with the composition containing one or more of the oligonucleotides of this invention. Then, dispensed bone marrow from another site of the animal (e.g., from the hip) is infiltrated into the mold, and the mold is placed into the jaw for final implantation.

Preferably, the device is treated with the composition containing one or more of the oligonucleotides of this invention (e.g. solution or gel) for a sufficient period of time to allow adsorption. Both the concentration of the oligonucleotides of this invention in the solution or gel and the time of exposure depend on a number of factors, including the volume of the defect and the nature of the site to which it is applied, and should be adjusted accordingly. As the size of the defect increases, or when the site is other than a bone site, the concentration of the oligonucleotides and the time of pre-soaking should be increased. The treatment should preferably be for at least about 0.5 hour, depending on the factors mentioned above (more preferably at least about 1 hour, and most preferably 1-2 hours), before implantation. Also depending on the above considerations, the concentration of oligonucleotides in the composition should preferably be of at least about 0.1 mg/ml (more preferably of at least about 0.5-10 and up to 100 mg/ml). The treatment may consist of any mode by which the composition is applied to the device to deliver effectively the osteogenic oligonucleotides of this invention and the osteogenic cell source if necessary. Such treatment includes, for example, adsorption or impregnation, depending in part on the nature of the indication. The compositions containing the osteogenic oligonucleotides of this invention to be used in the therapy will be dosed in a fashion consistent with good medical practice taking into account the nature of the skeletal tissue deficiency to be treated, the species of the host, the medical condition of the individual patient, the presence of any other drug in the composition, the site of delivery of the agent, the method of administration, the scheduling of administration, and other factors known to practitioners. Because of differences in host response, significant site-to-site and patient-to-patient variability exists. For purposes herein, the “therapeutically effective amount” of the osteogenic oligonucleotides of this invention is an amount that is effective to induce bone growth, as defined above, at the site of skeletal tissue deficiency.

As a general proposition, the osteogenic oligonucleotides of this invention are formulated and delivered to the target site at a dosage capable of establishing an oligonucleotide level equal or greater than about 0.1 mg/ml at the site. Typically, the oligonucleotide concentrations range from about 0.1 mg/ml to 12 mg/ml, preferably from about 1 to 4 mg/ml. These intra-tissue concentrations are maintained preferably by topical application and/or sustained release.

As noted above, these suggested amounts of oligonucleotides are subject to a great deal of therapeutic discretion. The key factor in selecting an appropriate dose and scheduling is the result obtained. Clinical parameters to determine an endpoint include increase in bone formation and mass and in radiographically detectable bone height. Such measurements are well known to those clinicians and pharmacologists skilled in the art.

The oligonucleotide composition is administered either locally to the site by any suitable means, including topical and continuous release formulation, or systemically, as needed. The oligonucleotides are generally combined at ambient temperature at the appropriate pH, and at the desired degree of purity, with a physiologically acceptable carrier, i.e., a carrier that is non-toxic to the patient at the dosages and concentrations employed. The carrier may take a wide variety of forms depending on the form of preparation desired for administration.

For the preparation of a liquid composition suitable for impregnation of a device, the carrier is a suitable buffer, a low molecular weight (less than about 10 residues) polypeptide, a protein, an amino acid, a carbohydrate (including glucose or dextrans), a chelating agent such as EDTA, cellulose, or other excipients. In addition, the oligonucleotide composition is preferably sterile. Sterility is readily accomplished by sterile filtration through 0.2 micron membranes. The oligonucleotide will be ordinarily stored as an aqueous solution, although lyophilized formulations for reconstitution are acceptable.

Generally, where the bone disorder allows so, one should formulate and dose the oligonucleotide for site-specific delivery, where the oligonucleotide is formulated into a sterile sustained-release composition suitable for local application to the desired site.

For local application of the oligonucleotide composition, for example, in the case of a bone defect that is a crack, e.g., a union fracture; the carrier may be any vehicle effective for this purpose. For obtaining a gel formulation, the liquid composition is typically mixed with an effective amount of a water-soluble polysaccharide, polyethylene glycol, or synthetic polymer such as polyvinylpyrrolidone to form a gel of the proper viscosity to be applied topically. The polysaccharide is generally present in a gel formulation in the range of 1-90% by weight of the gel, more preferably 1-20%. The polysaccharide that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; starch and fractionated starch, agar; alginic acid and alginates, gum arabic, pullullan, agarose, carrageenan, dextrans, dextrins, fructans, inulin, mannans, xylans, arabinans, chitosans, glycogens, glucans, and synthetic biopolymers, as well as gums such as xanthan gum, guar gum, locust bean gum, gum arabic, tragacanth gum, and karaya gum, and derivatives and mixtures thereof. The preferred gelling agent herein is one that is inert to biological systems, non-toxic, simple to prepare, not too runny or viscous, and one that will not destabilize the oligonucleotide held within it. Preferably the polysaccharide is an etherified cellulose derivative, more preferably one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. Most preferred herein is methylcellulose.

The polyethylene glycol useful for gelling is typically a mixture of low and high molecular weight polyethylene glycols to obtain the proper viscosity. For example, a mixture of a polyethylene glycol of molecular weight of 400-600 Dalton with one of molecular weight of 1,500 would be effective for this purpose when mixed in the proper ratio to obtain a paste. The term “water soluble” as applied to the polysaccharides and polyethylene glycols is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

In a preferred embodiment, the gel contains about 2-5% by weight methylcellulose and the oligonucleotide is present in an amount of about 10-1000 μg per ml of gel. More preferably, the gel consists of about 3% methylcellulose by weight, lactic acid to pH 5.0, and 20-200 μg per ml of oligonucleotide.

For the preparation of a sustained-release formulation, the oligonucleotide is suitably incorporated into a biodegradable matrix or microcapsular particle. A suitable material for this purpose is a polylactide, although other polymers of poly (.alpha.-hydroxycarboxylic acids), such as poly-D-(−)-3-hydroxybutyric acid (EP 133,988A), can be used. Additional biodegradable polymers include poly(lactones), poly(acetals), poly(orthoesters) or poly(orthocarbonates). The oligonucleotide is also suitably mixed with a biodegradable protein carrier such as collagen, atelocollagen, or gelatin to form a carrier matrix having sustained-release properties; the resultant mixture is then dried, and the dried material is formed into an appropriate shape, as described in U.S. Pat. No. 4,774,091.

The initial consideration here must be that the carrier itself, or its degradation products, are non-toxic in the target bone site and will not further aggravate the condition. This can be determined by routine screening in animal models of the target bone disorder or, if such models were unavailable, in normal animals. For examples of sustained-release compositions, see U.S. Pat. No. 3,773,919, EP 58,481A, U.S. Pat. No. 3,887,699, EP 158,277A, Canadian Patent No. 1176565, U. Sidman et al., Biopolymers, 22:547 (1983), and R. Langer et al., Chem. Tech., 12:98 (1982).

Controlled delivery of the oligonucleotide to a site is also suitably accomplished using permeable hollow cellulose acetate fibers with the oligonucleotide placed in the site and removed 24 hours later or left for longer periods of time (U.S. Pat. No. 4,175,326). Also, acrylic resin strips or cast films can be impregnated with the oligonucleotide and applied to the affected site. In addition, narrow dialysis tubing can be filled with a solution of the oligonucleotide and placed so as to deliver it to the appropriate site.

The composition herein may also suitably contain other osteogenetic factors such as IGF-I, TGF-beta 1, and PDGF. Such osteogenetic factors are suitably present in an amount that is effective for the intended purpose, i.e., to promote formation of bone.

The invention will be better understood by reference to the following examples. They should not, however, be construed as limiting the scope of the invention. All literature citations are incorporated by reference.

### Example 1

### Materials and Methods

The following materials and methods were generally used throughout the examples.

### 1) Oligonucleotides

Oligonucleotides having phosphorothioate internucleotide linkages were purchased, purified by high-pressure liquid chromatography (HPLC), from Operon Technologies (Alameda, Calif.) or Annovis (Aston, Pa.) or Oligos Etc (Bethel, Me.). ODNs were suspended in depyrogenated water, assayed for LPS contamination using the Limulus test and kept at −20° C. until used. Purity was assessed by HPLC and PAGE assays. ODN preparations were used if LPS levels were undetectable.

### 2) Animal Experiments

Young (8-12 weeks old) adult male Spragüe Dawley rats weighing about 350 g were used. Animals were anesthetized by intraperitoneal (i. p.) injection of a mixture of ketamine (50 mg/kg) and xilacine (5 mg/kg). After skin overlying the hind limb was shaved and sterilized. A 1.5 cm longitudinal incision was performed in the tibia forefront zone. To generate a defect in the bone, an osteotomy was made using a low-speed dental drill attached to a round diamond saw under saline irrigation. For the osteotomy, a site free of muscular insertions was selected 15 mm below the ankle. The wound was deep enough to reach the bone marrow. A single dose (as stated in each experiment) of ODN in 3 μl of methylcellulose 1% was introduced into the defect made in the right tibia. As a control, the same volume (3 μl) of vehicle was introduced into the defect made in the left tibia. Fracture callus formation was evaluated radiographically on days 0, 21 and 28. On day 28, animals were euthanized under ether atmosphere, and the tibias removed and photographed. After this, the tibias were fixed in 10% formol solution, decalcified in 10% EDTA solution, and embedded in paraffin. A longitudinal section of each tibia was cut, stained with hematoxilin/eosine and examined under a light microscope.

### Example 2

### Osteogenesis Stimulation by Oligonucleotides

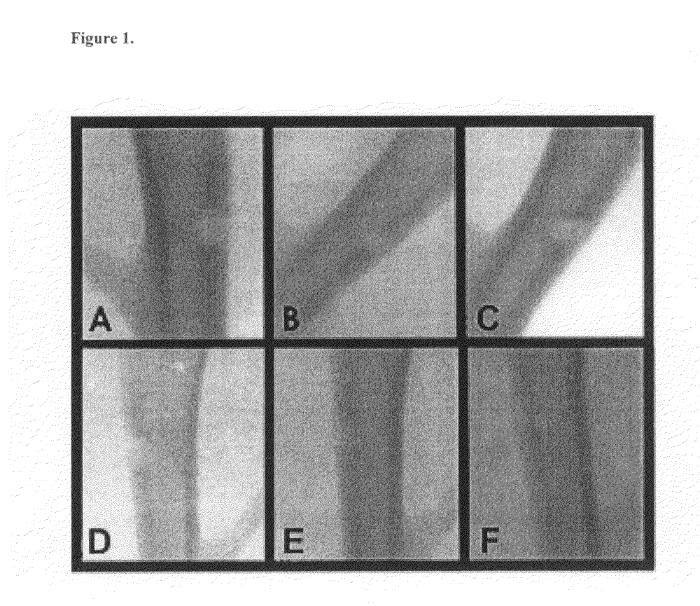

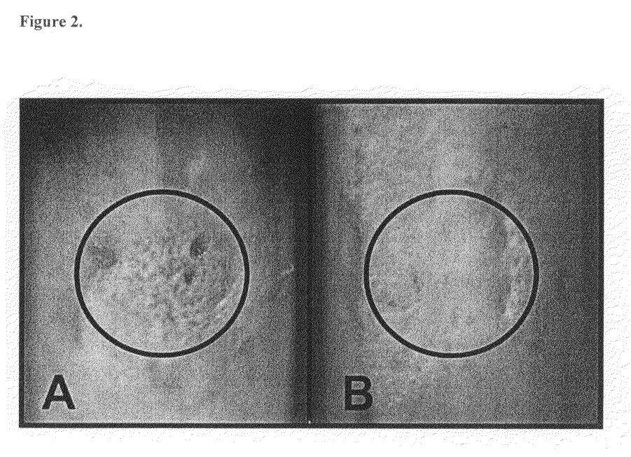

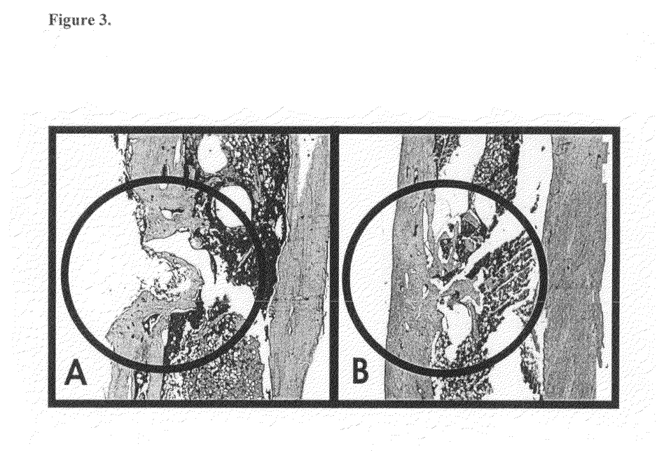

A rat femur tibial model was employed as described in Example 1. First, a total dose per defect of 60 μg of oligonucleotide was used. The oligonucleotide used in these experiments was IMT504. This oligonucleotide is 24 nucleotides long, its nucleotide sequence is 5′-TCATCATTTTGTCATTTTGTCATT-3′, and all the DNA (natural) phosphodiester bonds have been replaced with phosphorothioate bonds to protect it from enzymatic degradation. As example, FIG. 1 shows radiographs corresponding to both tibias of an experimental rat. In the right tibia, were the bond defect has been filled with vehicle plus IMT504, radiodense material filling the defect can be observed as early as 3 weeks after initiation of the treatment. In this treated right tibia, the defect is no longer visible at week 5 after initiation of the treatment. On the other hand, the defect is clearly visible by week 5 in the untreated (control) left tibia. FIG. 2 shows a photograph of the right (treated with IMT504) and left (untreated) tibia at the site where the experimental osteotomy was performed. As can be observed, the defect in the treated tibia appears completely filled with apparently normal bond while the defect in the control tibia is filled with spongy material corresponding to an incomplete ossification. The corresponding histological documentation can be seen in FIG. 3. In this figure the treated tibia shows well-formed bone tissue at the site where the osteotomy was performed. In contrast, in the untreated tibia the defect is still visible.

### Example 3

### Effect of the ODN Concentration in Osteogenesis Induction

In order to evaluate the minimal amount of IMT504 necessary to obtain rapid ossification, a rat femur tibial model was employed as described in Example 1. Table 1 shows that filling the osteotomy with a gel containing at least 0.4 mg/ml of IMT504 is necessary to obtain rapid ossification even though some activity was observed in a concentration as low as 0.06 mg/ml.

TABLE 1

| | | | | | | | | |
|---|---|---|---|---|---|---|---|---|
| IMT504 concentration | 0 | 0.06 | 0.4 | 0.8 | 1.5 | 3 | 6 | 12 |
| (mg/ml) | | | | | | | | |
| Ossification 35 days | − | +/− | + | + | + | + | + | + |
| after operation | | | | | | | | |

IMT504 (SEQ ID N^{o} 2)

### Example 4

### Induction of Osteogenesis by ODNs with Different Composition

Several of the components of the immune-system play a role in regulation of the osteogenic proccess (Shinoda K, Sugiyama E, Taki H, Harada S, Mino T, Maruyama M, Kobayashi M. Resting T cells negatively regulate osteoclast generation from peripheral blood monocytes. Bone. 2003 October; 33(4):711-20; Evans D B, Bunning R A, Russell R G. The effects of recombinant human granulocyte-macrophage colony-stimulating factor (rhGM-CSF) on human osteoblast-like cells. Biochem Biophys Res Commun. 1989 Apr. 28; 160(2):588-95; Fujikawa Y, Sabokbar A, Neale S D, Itonaga I, Torisu T, Athanasou N A. The effect of macrophage-colony stimulating factor and other humoral factors (interleukin-1, -3, -6, and -11, tumor necrosis factor-alpha, and granulocyte macrophage-colony stimulating factor) on human osteoclast formation from circulating cells. Bone. 2001 March; 28(3):261-7.). Therefore, we investigated the effect of different kinds of immunostimulatory ODNs in the osteogenetic process. Table 2 shows that the best stimulation of the osteogenesis was obtained using immunostimulatory ODNs of the PyNTTTTGT class (Elias F, Flo J, Lopez R A, Zorzopulos J, Montaner A, Rodriguez J M. Strong cytosine-guanosine-independent immunostimulation in humans and other primates by synthetic oligodeoxynucleotides with PyNTTTTGT motifs. J Immunol. 2003 Oct. 1; 171(7)3697-704.).

TABLE 2

| Oligo- | | Immunostimulatory | Osteogenic |
|---|---|---|---|
| nucleotide | Sequence | activity | activity |
| IMT022 | TGCTGCAA | − | +/−− |
| | AAGAGCAA | | |
| | AAGAGCAA | | |
| IMT518 | TCGTCGAA | +++ | +/−− |
| (CpG ODN) | AAGTCGAA | | |
| | AAGTCGAA | | |
| IMT504 | TCATCATT | +++ | +++ |
| (PyNTTTTGT | TTGTCATT | | |
| ODN) | TTGTCATT | | |

IMT022 (SEQ ID No 8);
IMT518 (SEQ ID No 106);
IMT504 (SEQ ID No 2)

These results indicate that ODNs with strong immunostimulatory activity are not necessarily strong stimulators of the osteogesis. However, the most active ODNs in osteogenesis are strongly immunostimulatory.

1. A use of an oligonucleotide having about 14 to 100 nucleotides for the manufacture of a medicament for inducing osteogenesis, characterized in that said oligonucleotide has at least one subsequence with the following composition: PyNTTTTNT or PyNTTTTGT, wherein Py is C or T and wherein N is any deoxyribonucleotide.

2. The use of an oligonucleotide according to claim 1 for the manufacture of a medicament for the treatment of osteogenic dysfunctions.

3. The use according to claim 1 for the manufacture of a medicament for the treatment of osteoporosis.

4. The use according to claim 1 for the manufacture of a medicament for the treatment of skeletal deficiency or alteration.

5. The use according to claim 1 characterized in that said oligonucleotide consists of 14 to 40 nucleotides.

6. The use according to claim 1 characterized in that said oligonucleotide has any kind of modification of the natural (phosphodiester) phosphate backbone.

7. The use according to claim 1 anyone of the above claims characterized in that said oligonucleotide has a phosphate backbone modification on the 5′ inter-nucleotide linkages.

8. The use according to claim 1 characterized in that said oligonucleotide has a phosphate backbone modification on the 3′ inter-nucleotide linkages.

9. The use according to claim 7 characterized in that at least one of the internucleotide linkages is a phosphorotioate linkage.

10. The use according to claim 1 characterized in that said oligonucleotide is selected from the group consisting of:

| | |
|---|---|
| TCATCATTTTGTCATTTTGTCATT; | (SEQ ID N^{o} 2) |
| TCCTCCTTTTGTCCTTTTGTCCTT; | (SEQ ID N^{o} 3) |
| TCTTCTTTTTGTCTTTTTGTCTTT; | (SEQ ID N^{o} 4) |
| TTGTTGTTTTGTTGTTTTGTTGTT; | (SEQ ID N^{o} 7) |
| TCATTTTTTTGTTTTTTTGTCATT; | (SEQ ID N^{o} 10) |
| TCATTGTTTTGTTGTTTTGTCATT; | (SEQ ID N^{o} 11) |
| TCATTCTTTTGTTCTTTTGTCATT; | (SEQ ID N^{o} 12) |
| TCATTATTTTGTTATTTTGTCATT; | (SEQ ID N^{o} 15) |
| TCATCCTTTTGTCCTTTTGTCATT; | (SEQ ID N^{o} 17) |
| TCATCTTTTTGTCTTTTTGTCATT; | (SEQ ID N^{o} 18) |
| CATTTTGTTTTTTTTTTTTTTTTT; | (SEQ ID N^{o} 20) |
| TTCATTTTGTTTTTTTTTTTTTTT; | (SEQ ID N^{o} 21) |
| TTTTCATTTTGTTTTTTTTTTTTT; | (SEQ ID N^{o} 22) |
| TTTTTTCATTTTGTTTTTTTTTTT; | (SEQ ID N^{o} 23) |
| TTTTTTTTCATTTTGTTTTTTTTT; | (SEQ ID N^{o} 24) |
| TTTTTTTTTTCATTTTGTTTTTTT; | (SEQ ID N^{o} 25) |
| TTTTTTTTTTTTCATTTTGTTTTT; | (SEQ ID N^{o} 26) |
| TTTTTTTTTTTTTTCATTTTGTTT; | (SEQ ID N^{o} 27) |
| TTTTTTTTTTTTTTTTCATTTTGT; | (SEQ ID N^{o} 28) |
| TTTTCATTTTGTCATTTTGTTTTT; | (SEQ ID N^{o} 29) |
| TCATCAATTTGTCAATTTGTCATT; | (SEQ ID N^{o} 30) |
| ACATCATTTTGTCATTTTGTCATT; | (SEQ ID N^{o} 46) |
| CCATCATTTTGTCATTTTGTCATT; | (SEQ ID N^{o} 47) |
| GCATCATTTTGTCATTTTGTCATT; | (SEQ ID N^{o} 48) |
| TAATCATTTTGTCATTTTGTCATT; | (SEQ ID N^{o} 49) |
| TTATCATTTTGTCATTTTGTCATT; | (SEQ ID N^{o} 50) |
| TGATCATTTTGTCATTTTGTCATT; | (SEQ ID N^{o} 51) |
| TCCTCATTTTGTCATTTTGTCATT; | (SEQ ID N^{o} 52) |
| TCTTCATTTTGTCATTTTGTCATT; | (SEQ ID N^{o} 53) |
| TCAACATTTTGTCATTTTGTCATT; | (SEQ ID N^{o} 55) |
| TCACCATTTTGTCATTTTGTCATT; | (SEQ ID N^{o} 56) |
| TCAGCATTTTGTCATTTTGTCATT; | (SEQ ID N^{o} 57) |
| TCATCATTTTGTCATTTTGTAATT; | (SEQ ID N^{o} 58) |
| TCATCATTTTGTCATTTTGTTATT; | (SEQ ID N^{o} 59) |
| TCATCATTTTGTCATTTTGTGATT; | (SEQ ID N^{o} 60) |
| TCATCATTTTGTCATTTTGTCCTT; | (SEQ ID N^{o} 61) |
| TCATCATTTTGTCATTTTGTCTTT; | (SEQ ID N^{o} 62) |
| TCATCATTTTGTCATTTTGTCAAT; | (SEQ ID N^{o} 64) |
| TCATCATTTTGTCATTTTGTCACT; | (SEQ ID N^{o} 65) |
| TCATCATTTTGTCATTTTGTCAGT; | (SEQ ID N⁰ 66) |
| TCATCATTTTGTCATTTTGTCATA; | (SEQ ID N^{o} 67) |
| TCATCATTTTGTCATTTTGTCATC; | (SEQ ID N^{o} 68) |
| TCATCATTTTGTCATTTTGTCATG; | (SEQ ID N^{o} 69) |
| TCATCAATTGGTCAATTGGTCATT; | (SEQ ID N^{o} 75) |
| TCATCAACTGGTCAACTGGTCATT; | (SEQ ID N^{o} 77) |
| TCATCATTGTGTCATTGTGTCATT; | (SEQ ID N^{o} 84) |
| TCATCATTTGGTCATTTGGTCATT; | (SEQ ID N^{o} 87) |
| TGCTGCTTTTGTGCTTTTGTGCTT; | (SEQ ID N^{o} 91) |
| TCATCATCTTGTCATCTTGTCATT; | (SEQ ID N^{o} 95) |
| TCATCATGTTGTCATGTTGTCATT; | (SEQ ID N^{o} 96) |
| GGGGGTCTTTTTTTCTTTTTTTTT | (SEQ ID N^{o} 107) |
| TTTTTTCTTTTTTTCTTTTTTGGG | (SEQ ID N^{o} 108) |
| AAAAATCTTTTTTTCTTTTTTTTT | (SEQ ID N^{o} 109) |
| TGCTGCTTTTATGCTTTTATGCTT | (SEQ ID N^{o} 110) |
| TCATCATTCTGTCATTCTGTCATT | (SEQ ID N^{o} 111) |
| TTTTTTCTTTTTTTCTTTTTTTTT | (SEQ ID N^{o} 112) |
| TGCTGCTTTTCTGCTTTTCTGCTT | (SEQ ID N^{o} 113) |
| TTTTTTCTTTTCTTTTTTTTTTTT | (SEQ ID N^{o} 114) |
| TTTTTCCTTTTTTCCTTTTTTTTT | (SEQ ID N^{o} 115) |
| CCCCCTCTTTTTTTCTTTTTTTTT | (SEQ ID N^{o} 116) |
| TTTTTTCTTTTTCTTTTTTTTTTT | (SEQ ID N^{o} 117) |
| TTTTTTCTTTTTTTCTTTTTTCCC | (SEQ ID N^{o} 118) |
| TTTTTTCTTTTTCTCTTTTTCTCT | (SEQ ID N^{o} 119) |
| TTTTTGCTTTTTTGCTTTTTTTTT | (SEQ ID N^{o} 120) |
| TTTTTACTTTTTTACTTTTTTTTT | (SEQ ID N^{o} 121) |
| TCATAATTTTGTAATTTTGTCATT | (SEQ ID N^{o} 122) |
| TTTTTTCTTTTTTTCTTTTTTAAA | (SEQ ID N^{o} 123) |
| TTTTTTCTTTTTTCTTTTTTTTTT | (SEQ ID N^{o} 124) |
| TTTTTTTTTTTTCATTTTGTGGGG | (SEQ ID N^{o} 125) |
| TTTTTTTTTTTTCATTTTGTTTTG | (SEQ ID N^{o} 126) |
| GGGTTTTTTTTTCATTTTGTTTTT | (SEQ ID N^{o} 127) |
| GTTTTTTTTTTTCATTTTGTTTTG | (SEQ ID N^{o} 128) |

11. The use according to claim 1 characterized in that said medicament is administered to a human.

12. The use according to claim 1 characterized in that said oligonucleotide is encapsulated in a slow release delivery vehicle.

13. The use according to claim 1 characterized in that said oligonucleotide is included in a pharmaceutically acceptable carrier.

14. The use according to claim 1 characterized in that said oligonucleotide is encapsulated in a slow release delivery vehicle.

15. The use according to claim 1 characterized in that said oligonucleotide osteogenic oligonucleotide is administered to a subject combined with a device aimed to aid in the treatment of a skeletal deficiency or alteration.

16. The use according to claim 15 characterized in that said device is a molded implant, a prosthetic device, a capsule, titanium alloy, or a ceramic block.

17. The use according to claim 16 characterized in that said device is a ceramic block comprising 0-100% hydroxylapatite and the remaining 100-0% tricalcium phosphate, by weight.

18. The use according to claim 16 characterized in that said medicament is combined with the device by adsorption, impregnation or inclusion into holes or channels performed in the device to hold the preparation.

19. The use according to claim 18 with the addition of an osteogenic cell source.

20. The use according to claim 1 characterized in that said oligonucleotide is present in amounts of 1 μg to 100 mg per dose.

21. The use according to claim 1 characterized in that said medicament is selected from the group consisting of liquid, gel and lyophilized formulations.

22. The use according to claim 1 characterized in that said medicament is administered by intradermic, intramuscular or intravenous injection.

23. The use according to claim 1 characterized in that said medicament is administered by an oral, intranasal, anal, vaginal, or trans-dermal route.
